(19) United States
(12) Patent Application Publication
(10) Pub. No.: US 2005/0079140 A1
(43) Pub. Date: Apr. 14, 2005

(54) Toothpaste formulation with green tea extract and fluoride

(76) Inventor: Kenneth H. Nussen, Santa Monica, CA (US)

Correspondence Address: BLAKELY SOKOLOFF TAYLOR & ZAFMAN, 12400 WILSHIRE BOULEVARD, SEVENTH FLOOR, LOS ANGELES, CA, 90025-1030, US

(21) Appl. No.: 10/792,963

(22) Filed: Mar. 3, 2004

Related U.S. Application Data
(63) Continuation-in-part of application No. 10/682,747, filed on Oct. 8, 2003.

(51) Int. Cl. A61K 7/16; A61K 7/18; A61K 7/26

(52) U.S. Cl. 424/49; 424/52; 424/58

(57) ABSTRACT

Toothpaste adapted to deliver active ingredients including green tea extract to the mouth of a consumer. Toothpaste may contain tea tree oil or fluoride. A natural toothpaste using natural colorings, flavorants, and bacterial agent and similar ingredients.

### CROSS-REFERENCE TO RELATED APPLICATION

The application is a Continuation-In-Part of co-pending application Ser. No. 10/682,747, filed Oct. 8, 2003, by applicant, Kenneth H. Nussen, entitled “Method and Apparatus for Oral Hygiene Products with Green Tea Extract.”

### BACKGROUND

1. Field

Embodiments of the invention relate to oral hygiene products utilizing green tea extract and tea tree oil. Specifically, embodiments of the invention relate to toothpaste utilizing all natural ingredients including green tea extract and fluoride.

2. Background

Oral hygiene products include toothpicks, dental floss, mouthwash, dental chewing gum, dental strips and toothpaste. Toothpaste is a substance used in conjunction with a toothbrush to clean teeth and protect teeth from cavities. Toothpaste includes cleansing agents to clean teeth and agents that assist in scrubbing away plaque build up on the surface of teeth.

Green tea extract is derived from the leaves of the plant Camellia sinensis and is rich in antioxidants. Green tea extract is commonly available in pill form as a nutritional supplement. Green tea extract has been shown to be effective at fighting cavities, prohibiting the growth of oral bacteria, and reducing inflammation of the gums.

### BRIEF DESCRIPTION OF THE DRAWINGS

The claims are illustrated by way of example and not by way of limitation in the figures of the accompanying drawings in which like references indicate similar elements. It should be noted that different references to “an” or “one” embodiment in this disclosure are not necessarily to the same embodiment, and such references mean at least one.

FIG. 1 is an illustration of toothpaste applied to a toothbrush.

FIG. 2 is an illustration of toothpaste packaged in a tube.

### DETAILED DESCRIPTION

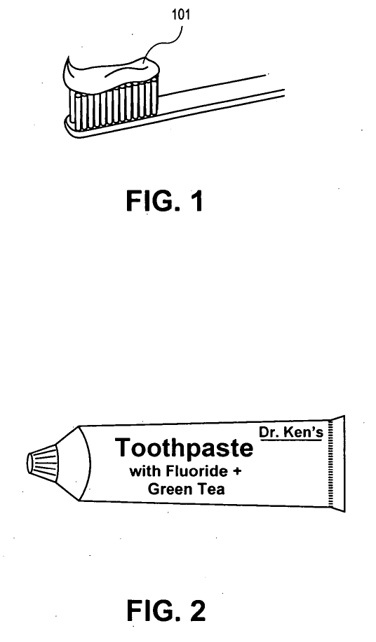

FIG. 1 is an illustration of a toothpaste that has anti-bacterial active ingredients and that is on the bristles of a toothbrush. Toothpaste 101 may be composed of abrasive agents, detergent, humectants, and thickeners. Toothpaste 101 may also contain preservatives, flavoring agents, sweeteners and coloring agents. These ingredients may be used to form a base composition that may be combined with an active ingredient, such as an amount of green tea extract (GTE) that is effective at reducing oral bacteria and fighting cavities. A suitable amount includes, but is not limited to a range of 1 to 50 percent by weight GTE in a batch. In a further embodiment, toothpaste 101 may be combined with fluoride to further protect teeth from decay.

In one embodiment, abrasive agents may be utilized in toothpaste 101 to remove stains and plaque build up on teeth. The abrasive agents may also serve to polish the surface of teeth to give the teeth a greater luster. Abrasive agents may include calcium phosphates, alumina, calcium carbonate, silica or similar materials and compositions. In one embodiment, toothpaste 101 contains a small amount of abrasive materials for use by individuals with sensitive teeth or to minimize damage to the enamel of teeth. In one embodiment, calcium carbonate (Vicron 15-15) may be utilized.

In one embodiment, toothpaste 101 may contain detergents. Detergents may be used to create a foam or froth when applied to teeth in the presence of water and an agitating motion. The foam may be used to assist an individual in distributing toothpaste 101 throughout the mouth of the individual and to maintain toothpaste 101 in the mouth of the user. In one embodiment, a detergent may be sodium lauryl sulfate, sodium lauryl sarcosinate hamposyl or similar materials or compounds.

In one embodiment, toothpaste 101 may contain humectants. Humectants may be used to provide a desired texture to toothpaste 101. Humectants may also be used to maintain moisture in toothpaste 101. Glycerin, sorbitol, xylitol, water or similar materials and compounds may be used as humectants in toothpaste 101. In one embodiment, natural kosher glycerin may be utilized. In one embodiment, purified water may be utilized to maintain the freshness of the toothpaste compoosition.

In one embodiment, toothpaste 101 may contain a thickener. A thickener may be used to provide body and texture to toothpaste 101. Toothpaste 101 may contain carrageenan, cellulose gum, xantham gum or similar materials and compositions. For example, carrageenan (seaweed) provides a smoothing texture to the toothpaste texture and also serves to sooth and protect gums.

In one embodiment, toothpaste 101 may contain fluoride. Fluoride may be used to strengthen the teeth of a user. Fluoride incorporates itself into tooth enamel making teeth more resistant to acids produce by plaque bacteria, as well as acids found in fruit juices, soft drinks and similar beverages and foods. Fluoride may be monofluorophosphate, stanous fluoride, sodium fluoride or similar materials and compositions.

In one embodiment, the toothpaste composition may include anti-tartar materials. Anti-tartar materials may include tetrapotassium pyrophosphate, tetrasodium pyrophosphate and similar anti-tartar materials and compounds. In one embodiment, anti-oxidants may also be utilized in the toothpaste composition. Aloe Vera gel may be used to provide anti-oxidant properties. Aloe Vera gel also serves to protect and soothe gums in contact with the toothpaste composition.

In one embodiment, flavoring (e.g., mint, cinnamon, bubble gum, anise, etc.), coloring, and sweeteners may optionally be added to the gum composition. In one embodiment, one or more sweeteners such as saccharin, fructose, sucrose, maltose, and corn syrup are added to the gum composition. The flavoring, coloring and sweeteners may be combined before being added to the gum composition. In one embodiment, mint, cinnamon, bubble gum, amount of approximately five percent by weight of the composition. Preservatives may also be utilized.

In one embodiment, the basic ingredients such as thickens and abrasive in the toothpaste composition may be mixed and hydrated to create a paste composition. In one embodiment, freeze-dried green tea extract (GTE) commercially available from XEL Herbaceuticals of Salt Lake City, Utah is added to the paste composition. In an embodiment where the paste also includes TTO, TTO liquid commercially available from Natural extracts of Australia of Los Angeles, Calif. is added to the gum composition.

In one embodiment, the toothpaste composition may include whiteners. Sodium Bicarbonate, papain (derived from a papaya plant) and similar whitening ingredients may be used. Sodium Bicarbonate also has anti-plaque properties.

The following table is an exemplary embodiment of the toothpaste composition:

Ingredients:
Green Tea Extract
Papain (Papaya Extract)
Sodim Bicarbonate
Vegatable Glycerin Kosher
Tetrapotassium Pyrophosphate
Tetrasodium Pyrophoaphate
Sodium Flouride
Aloe Vera gel
Polyol
Purified Water
Zeodent
Carrageenan
Natural Flavor (mint)
Sodium Lauroyl Sarcosinate Hamposyl.

In one embodiment, the toothpaste composition may be entirely or primarily formed of natural ingredients. Artificial sweetners, colorings and similar substances are not used. Anise, cinnamon, fruit concentrates, mint or similar naturally occurring spices and substances or combinations thereof may be used as flavorants. Anise oil also has anti-bacterial properties. Chlorophyll or similar substances may be used in the composition as a natural coloring. Chlorophyll and other natural coloring substances are derived from vegetables, plants, fruits and similar natural sources. For example, red coloring may be derived from grape skins, orange coloring from beta carotene and gray or white coloring from calcium carbonate. Tapioca starch may be used as natural water binding agent and texturizing agent. In one embodiment, stevia extract, xylitol, malitol, monoammonium glycyerrhizinate, or similar substances may be used as a sweetener. GTE is used in the all natural composition and provides anti-cavity, anti-bacterial, and anti-inflammatory properties.

The base composition and the active ingredients must be in amounts that are non-toxic and safe for human consumption. In one embodiment, the active ingredients may be combined and optionally hydrated before being added to the base toothpaste composition. The resulting toothpaste composition may then be transferred to a suitable container to retain moisture. A suitable container is nonporous and will not react chemically with the toothpaste composition. Representative packaging includes, but is not limited to, a squeezable tube (FIG. 2), a rigid tube, a pump, or similar packaging appropriate for pastes.

It is appreciated that in alternative embodiments various other toothpaste compositions can be formed to provide a desirable substance or substances to a user. For example, toothpaste can be formed that includes one or more vitamins that are necessary for or improve nutrition, or that act as coenzymes and precursors of coenzymes in the regulation of metabolic processes. Representative vitamins include, but are not limited to, Vitamin A, beta kerotene, Vitamin B, Vitamin B6, Vitamin B12, folic acid or folate, Vitamin C, Vitamin D, Vitamin E (including tocopherols) and or tocotrienols. Toothpaste may additionally or alternatively include minerals such as calcium, zinc, or similar materials. For example, paste may include an amount of a zinc compound such as zinc gluconate or other zinc compound in an amount sufficient to inhibit symptoms associated with a common cold. In one embodiment, the toothpaste includes up to 100 percent of the recommended daily allowance of one or more vitamins or minerals. In this manner, toothpaste may supplement the vitamins and minerals consumed by a user. Still further, the toothpaste may additionally or alternatively include various other herbs (e.g., herb extracts) such as ginko biloba, echinacea, kava kava or similar extracts.

The specification has been described with reference to specific embodiments thereof. It will, however be evident that various modifications and changes can be made thereto without departing from the broader spirit and scope of the appended claims. The specification and drawings are, accordingly, to be regarded in an illustrative rather than a restrictive sense.

1. A toothpaste comprising:
a thickening agent;
an abrasive agent;
fluoride; and
at least one of a vitamin, mineral and herb.

2. The toothpaste of claim 1, wherein the herb is a green tea extract.

3. The toothpaste of claim 1, further comprising:
a natural flavorant.

4. The toothpaste of claim 1, further comprising:
a natural coloring.

5. The toothpaste of claim 1, further comprising:
a natural sweetener.

6. The toothpaste of claim 4, wherein the natural coloring is chlorophyl.

7. The toothpaste of claim 5, wherein the natural sweetner is stevia extract.
